(19) United States
(12) Patent Application Publication
(10) Pub. No.: US 2004/0235062 A1
(43) Pub. Date: Nov. 25, 2004

(54) Method of analyzing protein occurring in cell or substance interacting with the protein

(76) Inventors: Terumi Nakajima, Tokyo (JP); Hideo Naoki, Osaka (JP); Akikazu Yasuda, Osaka (JP); Keiko Iwasa, Kyoto (JP)

Correspondence Address: MANELLI DENISON & SELTER, 2000 M STREET NW SUITE 700, WASHINGTON, DC, 20036-3307, US

(21) Appl. No.: 10/481,011

(22) PCT Filed: Jun. 19, 2002
(86) PCT No.: PCT/JP02/06103

(30) Foreign Application Priority Data
Jun. 19, 2002 (JP) 200-185602

(51) Int. Cl. G01N 33/53

(52) U.S. Cl. 435/7.2

(57) ABSTRACT

A method is disclosed for identifying or analyzing, easily and in a short time, the molecular weight, the chemical species of molecules, and the structure of molecules, of a protein present on the superficial layer of cells or in cells, or a substance interacting with the protein. This method comprises the steps of: bringing the cells having the protein and the substance interacting with the protein into contact with each other to cause the substance to interact with the cells; purifying the cells, with the cells and the substance in an interacting state; irradiating the purified cells with a laser to ionize the substance directly; and analyzing the ionized substance by mass spectrometry.

This method is a new method for screening a substance interacting with a protein present in cells, and a new method for determining the presence of a particular protein in cells with the use of a substance interacting with the protein, the new methods not relying on conventional bioassays. These methods are easy to operate and capable of accurate and prompt determination.

### TECHNICAL FIELD

This invention relates to a method for evaluating, identifying, and structurally analyzing substances interacting with proteins present in cells of organisms, and also relates to a method for determining the presence of particular proteins in cells of organisms by use of substances interacting with the proteins.

### BACKGROUND ART

On the superficial layer of cells or in cells (herein, both situations may be simply expressed as “in cells” collectively), various proteins are known to exist which play various roles in a living organism, such as a role as receptors, a role as ion channels, a role as transporters, a role as antibodies, a role as enzymes, and a role involved in intracellular signal transduction. It is also known that there are numerous substances which interact with these proteins to activate or inhibit the functions of these proteins, or to act as antigens for the antibodies, or to work as substrates for the enzymes.

However, not all has been elucidated about such proteins present in cells of organisms or such substances interacting with the proteins. Even nowadays, searches for new proteins, or for substances interacting with various intracellular proteins, have been conducted energetically by researchers all over the world.

To search the natural world for novel physiologically active substances interacting with particular proteins, bioassays using proteins with known activities have hitherto been employed generally. However, bioassays have included at least two problems. First, materials are consumed during purification of physiologically active substances, so that large amounts of the starting ingredient and naturally occurring matter (including organisms) for extraction of the ingredient are needed to secure sufficient amounts of materials for structural determination. Secondly, available bioassays are limited and, with the standard bioassays, many novel physiologically active substances may remain undetected.

Recently, matrix-assisted laser desorption/ionization time-of-flight mass spectrometry (MALDI-TOF MS) has become a powerful tool for the direct analysis of peptide profiles from small pieces of dissected tissues or from isolated single cells (Garden et al., 1996; De With et al., 1997; Garden et al., 1998; Redeker et al., 1998). In these experiments, it is possible to analyze very fine regions of samples, and the resulting peptide fingerprinting shows the biosynthesis and expression of bioactive peptides.

On the other hand, on-line capillary reversed-phase high performance liquid chromatography with mass spectrometric detection (capillary HPLC/MS and capillary HPLC/MS/MS) is available in modern bioanalysis. This tool provides excellent information on the molecular weights of peptides in samples, and the MS/MS system allows the peptides to be fragmented to yield fragment ions, enabling their amino acid sequences to be deduced.

There has been a desire for the development of a new method for screening substances interacting with proteins present in cells, and a new method for determining the presence of particular proteins in cells with the use of substances interacting with the proteins, the new methods not relying on conventional bioassays. As these methods, methods easy to operate and capable of accurate and prompt determination have been desired.

### DISCLOSURE OF THE INVENTION

We, the inventors of the present invention, already developed a method for directly identifying the chemical structure of a substance present in tissue or cells of organisms by use of MALDI-TOF MS, and filed an application for it as Japanese Patent Application No. 2000-060754. The present invention has been accomplished as a result of in-depth studies with a view to expanding the range of usage of this application. The present invention has succeeded in developing a method for determining the presence of a protein, which is present on a cell membrane or in cells, or the presence of a substance interacting with this protein, by the use of the protein and the substance.

### BRIEF DESCRIPTION OF THE DRAWINGS

FIG. 1 is a chart showing the results of MALDI-TOF-MS measurement of a ligand(hANP)-bound membrane fraction prepared with the use of cells having expressed GC-A, receptors of hANP, in Example 1.

FIG. 2 is a chart showing the results of MALDI-TOF-MS measurement of a ligand(hANP)-bound membrane fraction prepared with the use of blank cells free from expression of GC-A in Example 1.

FIG. 3 is a chart showing the results of MALDI-TOF-MS measurement of a ligand-bound membrane fraction prepared with the use of a ligand (a peptide mixture solution comprising four peptides including hANP) and GC-A expressed cells in Example 2.

FIG. 4 is a chart showing the results of MALDI-TOF-MS measurement of a ligand-bound membrane fraction prepared with the use of a ligand (a peptide mixture solution comprising four peptides including hANP) and blank cells in Example 2.

FIG. 5 is a chart showing the results of MALDI-TOF-MS measurement of only the peptide mixture solution used in Example 2.

### EMBODIMENTS OF THE INVENTION

A first embodiment of the method according to the present invention is that if a known protein is present on a cell membrane or in cells, the cells and an unknown test substance are brought into contact, causing the test substance to interact with the cells in some form, such as adsorption, binding or association. In this state, the cell or a slice of the cell (for example, a cell membrane) is purified, and the amount of the test substance present is confirmed by the use of MALDI-TOF MS. By this procedure, whether the test substance interacts with the known protein can be judged.

A second embodiment of the method according to the present invention is a method which is contrary to the first embodiment, and is the method which uses a known substance, for example, a ligand to a particular receptor, and judges whether an unknown protein present on a cell membrane or in cells is existent or not. In this case, the cells having the unknown protein are brought into contact with the known substance. If the known substance has interacted with the cells in some form, such as adsorption, binding or association, it can be judged that the protein, which the known substance acts on, is present on the cell membrane or in the cells.

Furthermore, the amino acid sequence of the protein, whose existence has been confirmed, can be determined by the method of the present invention.

According to the present invention, ligands, which act on various receptors expressed on the nuclear membrane and mitochondrial membrane as well as the cell membrane, are purified while being adsorbed to the membrane, and the ligand-bound cell membrane is irradiated with a laser beam. By this procedure, only the ligand can be directly ionized, and the ligand can be analyzed by mass spectrometry.

Examples of the receptors are peptide hormone receptors, ion channels, transporters and, further, antibodies expressed in lymphocytes and macrophages. The ligands, on the other hand, include not only various naturally occurring physiologically active substances, such as peptides, amino acids, amines, and steroids, but also synthetic compounds working as ligands. Thus, the present method is effective as a means of searching for unknown ligands interacting with such receptors, and can be used to search for receptors on the cell membrane which have unknown functions.

Mass spectrometry for use in the analysis system can make an analysis with a cell membrane fraction corresponding to 10⁴ to 10⁵ cells, for example, in the case of cells, or with a very small amount of a sample measuring 2 mm×2 mm×0.1 mm in thickness, for example, in the case of a tissue slice. Also, the mass spectrometry method can obtain, simultaneously and in a short time, molecular information on the molecular weight, the types of molecules (types, such as peptide proteins, steroids, lignans, catechins, and sugars; hereinafter referred to as “chemical species”), and the structure of molecules. That is, the use of the MALDI-TOF model as a mass spectrometer results in the measurement of the molecular weight of the ligand. Moreover, the full use of a R-column LC-ESI-QTOF-MS/MS analyzer, with its molecule related ions as precursor ions, enables the chemical species of adsorbed molecules and even their structure to be analyzed.

By combining these methods, the presence of novel physiologically active substances, or receptors expressed in cultured cells from tissue, can be predicted. These methods can contribute, particularly, to the discovery of orphan receptors and ligands thereto, and are also useful as a means of screening new medicines, or are useful for searching for disease markers.

According to the present invention, moreover, the method of the invention can be applied widely not only to combinations of receptors and ligands, but also to substances interacting with proteins in some form. These interactions include, for example, actions on proteins which are ion channels, transporters, antibodies, and enzymes. Substances having such actions include antigens to antibodies, substrates to enzymes, or activators or inhibitors.

The present invention will be described with reference, for example, to an embodiment in which a ligand to cells expressing a known receptor is detected and identified.

Known cells expressing known receptors are cultured to express the receptors. The cultured cells are used as sample cells, and treated in the manner described below. For comparison, the same type of cells which have not expressed the receptors are used. The present invention can utilize not only cultured cells expressing such known receptors, but also tissue cells from organ tissues, and dissociated cells such as lymphocytes and intraperitoneal monocytes.

Distilled water in a proper amount, for example, in an amount of 100 to 800 μl, is added to a suitable number, for example, 10³ to 10⁵, preferably 10⁴ to 10⁵, of the sample cells. The mixture is incubated at room temperature for 3 to 30 minutes, preferably 5 to 20 minutes, to crush the cells. The system is centrifuged to precipitate the cell membranes, or the system is filtered to separate the cell membranes. The cell membranes are washed with distilled water, and centrifuged or filtered again to remove the cytoplasm, thereby obtaining a receptor-expressed cell membrane fraction.

To the resulting membrane fraction, a solution of a substance, which is enough to coat a nonspecific adsorption portion of the membrane fraction and which has the effect of hindering the nonspecific adsorption of a substance, for example, 1 pmol/μl of albumin (BSA), is added in an amount of 200 to 500 μl, and the system is dispersed. The dispersion is incubated at room temperature for 3 to 30 minutes, preferably 5 to 20 minutes to coat the nonspecific adsorption portion of the membrane fraction with albumin. The membrane fraction is gathered by centrifugation or filtration, and washed with distilled water to remove the excess albumin.

A ligand in a sufficient amount to bind to the receptors, for example, 100 μl of a 10 pmol/μl ligand solution, μs added to the washed albumin-coated membrane fraction, and the mixture is incubated at room temperature for 3 to 30 minutes, preferably 5 to 20 minutes. Then, the system is subjected to a washing treatment involving washing with distilled water, followed by centrifugation (or filtration). This washing treatment is repeated two times or more, preferably 4 to 5 times, to remove fractions which have not adsorbed the excess ligand.

A proper amount, for example, 5 to 20 μl, of distilled water is added to the resulting ligand-adsorbed cell membranes, and the system is suspended by a voltex. The suspension (0.5 to 3 μl) is dispensed into a plate for MALDI, and air-dried. An α-cyano-4-hydroxycinnamic acid (αCHCA) matrix solution is added to the air-dried suspension, and MALDI-TOF-MS measurement is made. This measurement is made similarly for the comparative cell membrane which has not expressed the receptors. The results of these measurements are compared, whereby the ligand bound to the known receptors can be identified, with the molecular weight as an indicator.

According to the present invention, the full use of a μ-column LC-ESI-QTOF-MS/MS meter—using laser-ionized molecule related ions, which have been used for mass spectrometry, as precursor ions—enables the chemical species of the adsorbed molecules and even their structure to be analyzed.

According to the present invention, a protein present on the superficial layer of cells or in cells, or a substance interacting with the protein can be detected, identified or analyzed by the steps described below.

The above object can be achieved by a method comprising:

(1) the step of bringing cells into contact with a substance interacting with a protein on the superficial layer of the cells or in the cells to cause the substance to interact with the cells;

(2) the step of purifying the cells, with the cells and the substance being in an interacting state;

(3) the step of irradiating the purified cells with a laser, with the substance in the interacting state, to ionize the substance directly; and

(4) the step of analyzing the ionized substance by mass spectrometry.

The present invention can also screen a substance interacting with a protein present on the superficial layer of cells or in cells, the protein having known activity, by a method comprising the steps described below.

A mass spectrum obtained by

(1) the step of bringing a test substance into contact with cells, where a known protein is known to be present, to cause interaction;

(2) the step of purifying the cells having interacted with the test substance, or a fraction thereof;

(3) the step of irradiating the purified cells or the fraction thereof with a laser, thereby directly ionizing the substance having interacted with the cells; and

(4) the step of analyzing the ionized substance by mass spectrometry,

is compared with a mass spectrum obtained by performing the steps (1) to (4) with the use of cells where the protein has been found not to be present. If the former mass spectrum shows that the amount of the test substance increases, it can be judged that the test substance is a substance interacting with the protein. The identification of the substance makes it possible to screen and identify the substance interacting with the known protein present on the superficial layer of cells or in cells.

Furthermore, according to the present invention, expression cells or expression tissue for receptors interacting with a known ligand, for example, can be screened by the method described below.

This method comprises comparing a mass spectrum, which has been obtained by

(1) the step of bringing a known ligand into contact with tissue or cells, where the presence or absence of receptors to the ligand is to be confirmed, to cause interaction;

(2) the step of purifying the tissue or cells contacted with the ligand;

(3) the step of irradiating the purified tissue or cells with a laser, thereby directly ionizing the ligand present; and

(4) the step of analyzing the ionized ligand by mass spectrometry, with a mass spectrum obtained by performing only the step (2) and the subsequent steps, without performing step (1) in which the known ligand is brought into contact with the tissue or cells. If the former mass spectrum shows that the amount of the ligand increases, it can be judged that the receptors interacting with the ligand are present in the tissue or cells.

The present invention will be described in further detail with reference to the Examples offered below.

### EXAMPLE 1

### Experiments on Binding Between Human Atrial Natriuretic Peptide (Hereinafter Referred to as “hANP”) and hANP Receptor GC-A

Cultured cells were receptor expressed cells and blank cells. CHO/GC-A, i.e. CHO cells (Chinese hamster ovary cells) having expressed hANP receptor GC-A, were used as the receptor expressed cells. CHO host cells were used as the blank cells. MALDI-TOF-MS measurement samples were prepared by the same method in the following manner:

Step 1. Preparation of Membrane Fraction

Distilled water (500 μl) was added to 7.5×10⁵ cultured cells (GC-A expressed cells and blank cells), and the mixture was incubated at room temperature for 10 minutes. The resulting suspension was centrifuged to obtain pellets. Distilled water (500 μl) was further added to the pellets, and the mixture was centrifuged again, followed by removing the supernatant, to obtain a membrane fraction.

Step 2. Albumin Coating

To the resulting membrane fraction, 500 μl of a 1 pmol/μl aqueous albumin solution was added, and the mixture was incubated for 10 minutes at room temperature. The resulting suspension was centrifuged to obtain pellets. Distilled water (500 μl) was further added to the pellets, and the mixture was centrifuged again to obtain an albumin-coated membrane fraction.

Step 3. Binding to Ligand (hANP)

To the resulting albumin-coated membrane fraction, 100 μl of a 10 pmol/μl aqueous hANP solution was added, and the mixture was incubated for 10 minutes at room temperature. The resulting suspension was centrifuged to obtain pellets. The resulting pellets were further washed with 500 μl of distilled water, and centrifugation was performed again to obtain a ligand(hANP)-bound membrane fraction.

Step 4. MALDI-TOF-MS Measurement

Distilled water (10 μl) was added to the resulting ligand-bound membrane fraction, and the mixture was suspended. The suspension (1 μl) was dispensed into a plate for analysis, and air-dried. Then, 1 μl of an α-cyano-4-hydroxycinnamic acid (α-CHCA) saturated solution (methanol:water=1:1, 0.1% trifluoroacetic acid) was added to the air-dried suspension, and MALDI-TOF-MS measurement (Voyager Elite) was made.

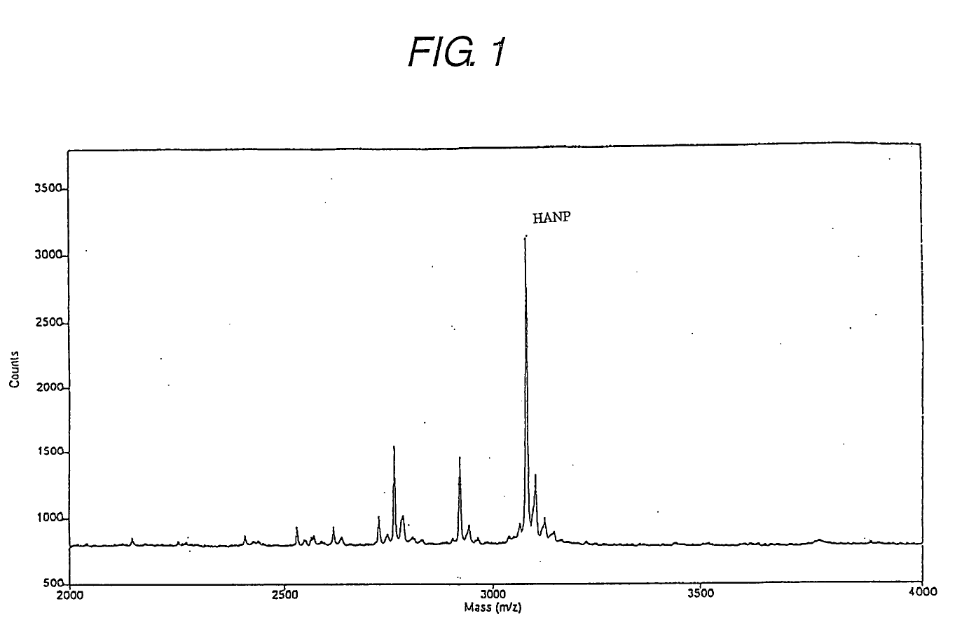

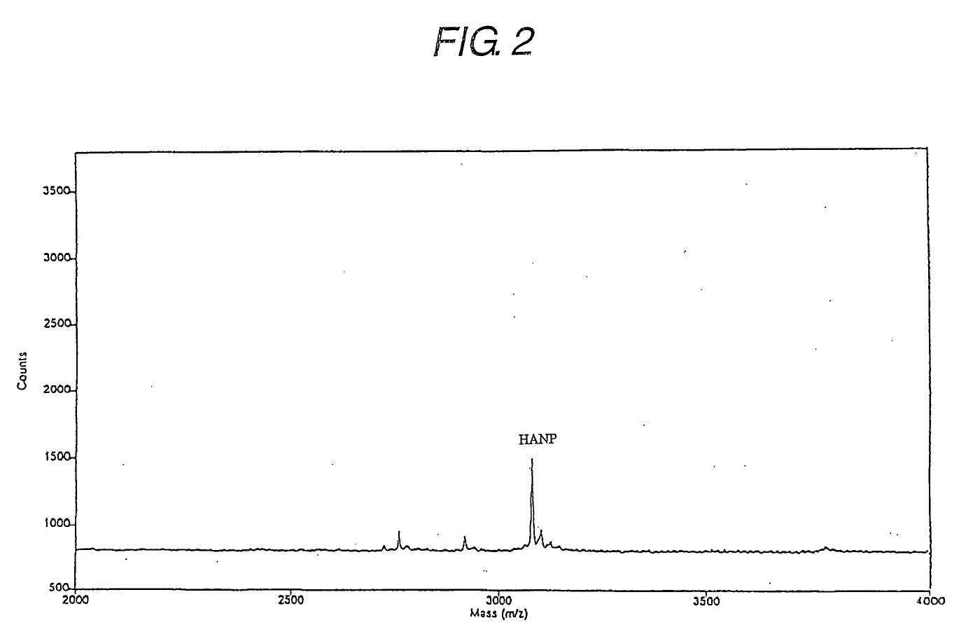

As shown in FIGS. 1 and 2, hANP detected was about three times as much in the GC-A expressed cells (FIG. 1) as in the blank cells (FIG. 2). From these findings, hANP can be judged to interact with GC-A receptors.

### EXAMPLE 2

### Experiments on Selectivity

Step 1. Preparation of Membrane Fraction and

Step 2. Albumin Coating

An albumin-coated membrane fraction (CHO/GC-A cells and CHO host cells) was obtained in the same manner as in Step 1. and Step 2. of Example 1.

Step 3. Binding to Ligand (Peptide Mixture)

To the resulting albumin-coated membrane fraction, 100 μl of a peptide mixture solution, i.e., an aqueous solution of a mixture of 10 pmol/μl of hANP, 10 pmol/μl of angiotensin I, 10 pmol/μl of substance P(SP), and 10 pmol/μl of melanocyte-stimulating hormone (α-MSH), was added, and the mixture was incubated for 10 minutes at room temperature. The resulting suspension was centrifuged to obtain pellets. The resulting pellets were further washed with 500 μl of distilled water, and centrifugation was performed again to obtain a ligand-bound membrane fraction.

Step 4. MALDI-TOF-MS Measurement

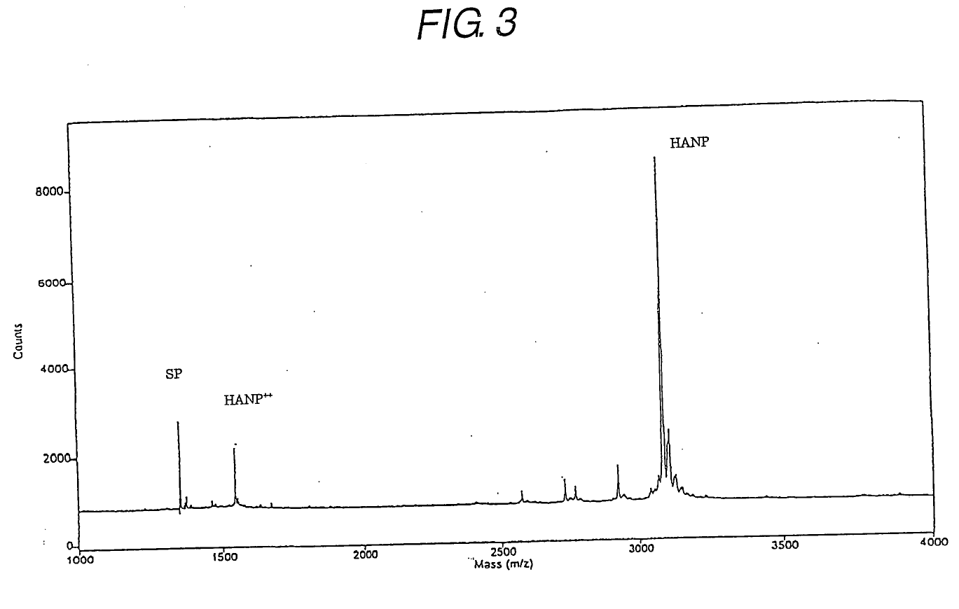

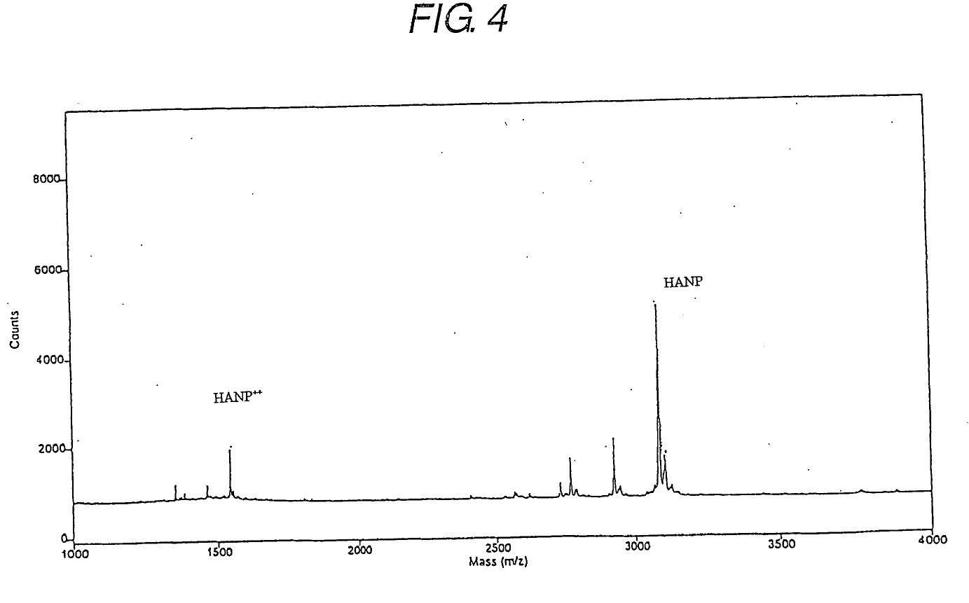

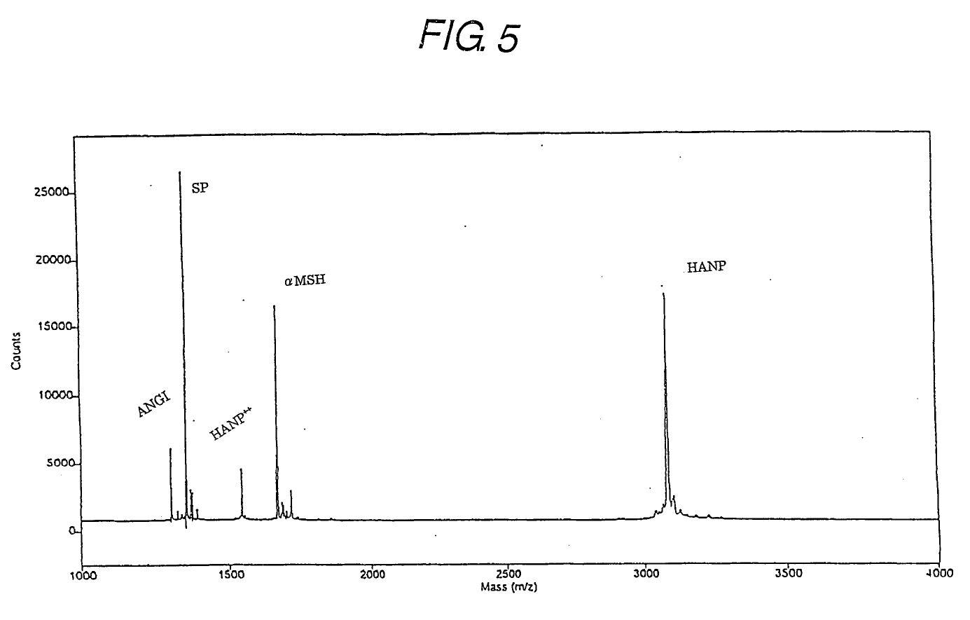

MALDI-TOF-MS measurement was made in the same manner as in Step 4. of Example 1. The results are shown in FIG. 3 (GC-A receptors were expressed) and FIG. 4 (the blank without expression of GC-A receptors). For comparison, MALDI-TOF-MS measurement was made only of 1 l of the peptide mixture solution (10 pmol/μl of hANP, 10 pmol/μl of angiotensin I, 10 pmol/μl of SP, and 10 pmol/μl of α-MSH) (FIG. 5).

It was found that hANP was bound to the GC-A receptor expressed cell membrane fraction 5 or more times as selectively as the three other peptides. Thus, it can be judged that only hANP interacts with GC-A receptors, and the other peptides, i.e. angiotensin I, substance P and α-MSH, do not interact with GC-A receptors.

### INDUSTRIAL APPLICABILITY

According to the method of the present invention, if a known protein is present on a cell membrane or in cells, the cells and an unknown test substance are brought into contact, causing the test substance to interact with the cells in some form, such as adsorption, binding or association. In this state, the cells or slices of the cell (for example, cell membranes) are purified, and the amount of the test substance present is confirmed by the use of MALDI-TOF MS. By this procedure, whether the test substance interacts with the known protein can be judged.

Another embodiment of the present invention is a method which is contrary to the above method, and which uses a known substance, for example, a ligand to a particular receptor, and judges whether an unknown protein present on a cell membrane or in cells is existent or not. In this case, the cells having the unknown protein are brought into contact with the known substance. If the known substance has interacted with the cells in some form, such as adsorption, binding or association, it can be judged that the protein, which the known substance acts on, is present on the cell membrane or in the cells. Furthermore, the amino acid sequence of the protein, whose existence has been confirmed, can be determined by the method of the present invention.

These methods are easy to operate, and capable of accurate and prompt judgment. Thus, a wide range of applications can be expected of them.

1. A method for identifying or analyzing a protein present on a superficial layer of cells or in cells, or a substance interacting with said protein, comprising:
(1) a step of bringing the cells having said protein and said substance interacting with said protein into contact with each other to cause said substance to interact with the cells;
(2) a step of purifying the cells, with the cells and said substance being in an interacting state;
(3) a step of irradiating the purified cells with a laser, with said substance being in the interacting state, to ionize said substance directly; and
(4) a step of analyzing the ionized substance by mass spectrometry.

2. The method according to claim 1, wherein said protein present on the superficial layer of cells or in the cells is a protein showing a physiological activity as a receptor, an ion channel, a transporter, an antibody, or an enzyme.

3. The method according to claim 1, wherein said substance interacting with said protein is a ligand, an antigen or a substrate to said protein.

4. The method according to claim 1, wherein said substance interacting with said protein is a substance present in a living organism.

5. The method according to claim 4, wherein said substance present in a living organism has a molecular weight of about 500 to about 3,000.

6. The method according to claim 1, wherein said substance interacting with said protein is a substance not present in a living organism.

7. The method according to claim 1, wherein said mass spectrometry is matrix-assisted laser desorption/ionization time-of-flight mass spectrometry (MALDI-TOF MS).

8. The method according to claim 7, wherein said mass spectrometry is a method using a matrix-assisted laser desorption/ionization time-of-flight mass spectrometer (MALDI-TOF MS) and a microcolumn liquid chromatography electrospray ionization quadrupole time-of-flight tandem mass spectrometer (μ-column LC-ESI-QTOF-MS/MS).

9. A method for screening a substance interacting with a protein present on a superficial layer of cells or in cells, said protein having known activity, said method comprising: comparing a mass spectrum obtained by
(1) a step of bringing a test substance into contact with cells, where said protein is known to be present, to cause interaction,
(2) a step of purifying the cells, with the cells and said test substance being in an interacting state,
(3) a step of irradiating the purified cells with a laser, thereby directly ionizing said substance interacting with the cells, and
(4) a step of analyzing the ionized substance by mass spectrometry, with a mass spectrum obtained by performing the steps (1) to (4) with use of cells where said protein has been known to absent, whereby presence of said substance interacting with said protein is determined.

10. The method according to claim 9, wherein said known protein is a receptor protein.

11. The method according to claim 10, wherein an isolated and established strain is used as the cells in which said receptor is expressed.

12. The method according to claim 10, wherein cells artificially forced to express said receptor are used as the cells in which said receptor is expressed.

13. The method according to claim 10, wherein orphan receptor expressing cells are used as the cells in which said receptor is expressed.

14. A method for screening cells or tissue, in which said protein is expressed, interacting with a substance with known activity, comprising:
comparing a mass spectrum obtained by
a step of bringing said substance into contact with tissue or cells, where presence or absence of said protein is to be confirmed, to cause interaction,
(2) a step of purifying the tissue or cells contacted with said substance,
(3) a step of irradiating the purified tissue or cells with a laser, thereby directly ionizing said substance present, and
(4) a step of analyzing the ionized substance by mass spectrometry, with a mass spectrum obtained by performing only the steps (2) to (4), without performing step (1) in which the ligand is brought into contact with said tissue or cells, whereby presence of said protein interacting with said substance is determined.

15. The method according to claim 14, wherein said substance is a ligand.

16. The method according to claim 14 or 15, wherein said protein is a receptor protein.
